(19) United States
(12) Patent Application Publication
(10) Pub. No.: US 2006/0269610 A1
(43) Pub. Date: Nov. 30, 2006

(54) Method for producing water dispersible dry powders from poorly soluble compounds

(76) Inventors: Jörg Rosenberg, Ellerstadt (DE); Thomas Hantke, Heidelberg (DE); Jörg Breitenbach, Mannheim (DE)

Correspondence Address: NOVAK DRUCE DELUCA & QUIGG, LLP, 1300 EYE STREET NW, SUITE 400 EAST TOWER, WASHINGTON, DC, 20005, US

(21) Appl. No.: 10/501,875

(22) PCT Filed: Feb. 7, 2003
(86) PCT No.: PCT/EP03/01244

§ 371 Date: Jul. 22, 2005

(30) Foreign Application Priority Data
Feb. 8, 2002 (DE) 102 05 362.6

(51) Int. Cl. A61K 9/14

(52) U.S. Cl. 424/489

(57) ABSTRACT

The invention relates to a method for producing water dispersible dry powders from hardly soluble compounds, whereby a dispersion is provided, containing the poorly soluble compound in a microdispersed form in a dispersion agent. The dispersion of the poorly soluble compound is concentrated by tangential-filtration and the dispersion agent is removed. The invention also relates to preparations based on said water dispersible dry powders.

The present invention relates to a process for producing water-dispersible dry powders of compounds which are poorly soluble or insoluble in water and to preparations based on such water-dispersible dry powders.

Numerous compounds are poorly soluble or insoluble in water but ought nevertheless to be used in an aqueous medium. Examples thereof are certain active pharmaceutical ingredients, food additives and cosmetic ingredients. It is therefore necessary to find procedures for dissolving such compounds sufficiently well in aqueous systems because, otherwise, their efficacy is greatly impaired. Poorly water-soluble active pharmaceutical ingredients are inadequately absorbed in the gastrointestinal tract after oral administration, and in the case of coloring agents, e.g. carotenoids for coloring human foods and animal feeds, only a low color yield is achieved. Various possibilities are already known for improving the solubilization of the compounds in aqueous media, e.g. reducing the particle size of the poorly soluble substances.

In order to achieve properties, e.g. absorption or coloring properties, which come as close as possible to the ideal state of molecular dispersion of the poorly soluble compounds, it is necessary for the poorly soluble compounds to be dispersed as finely as possible in the aqueous medium. A particle size of less than 1 μm is desirable in this connection. Such particle sizes can be achieved by grinding either not at all or only with harm to the compounds. Attempts have been made with carotenoids first to dissolve them using a water-soluble solubilizer and then to precipitate them as microcrystals by dilution with water. However, this has been thwarted to date by the solubility of the carotenoids in such solvents being too low.

Another possibility is to add solubilizing auxiliaries. Examples of suitable solubilizing auxiliaries are surfactants, alcohols, ethers, esters, etc., for pharmaceuticals especially the solubilizers monographed in the international pharmacopoeias. It is possible with such solubilizers in many cases to achieve micellar solubilization, i.e. the poorly soluble compound is attached to surfactant micelles or incorporated in them. However, it is necessary in many cases to employ rather large quantities of these solubilizers for the poorly soluble active ingredients. In the case of pharmaceuticals, this may cause unwanted side effects after oral administration of such active ingredient preparations.

A further possibility for bringing poorly soluble compounds into an optimally useful form is to prepare a colloidal solution of the relevant compound in water. In this case, the compound is incorporated into colloidal aggregates which can be produced from so-called protective colloids in water. Examples of such protective colloids are gelatin and/or casein.

Chimia 21,329 (1967), and DE-AS 12 11 911 and DE-OS 25 34 091, disclose processes in which the active ingredient is dissolved in a water-immiscible solvent, preferably a chlorinated hydrocarbon, the solution is emulsified by homogenization in a gelatin/sugar solution, and finally the solvent is stripped off from the emulsion, releasing the active ingredient in microcrystalline form. A finely divided powder can be obtained by removing water from the resulting suspension. The use of chlorinated hydrocarbons represents a serious disadvantage of this process, however.

Other processes for producing a product with finely dispersed active ingredients are the application of the active ingredients to carrier materials such as starch, pectin or dry milk powder, in which case for example a solution of the active ingredient in oil according to DE-PS 642 307 or chloroform according to DE-PS 361 637 and CH-PS 304 023 is sprayed on to the carrier materials. The resulting products are, however, not universally dispersible in aqueous media and have inadequate storage stability.

Chimia 21,329 (1967) and FR-PS 1 056 114, and U.S. Pat. No. 2,650,895, describe processes in which active ingredients in the form of their oily solutions are embedded emulsion-like in colloids such as gelatin. The active ingredient concentrations in the products produced in this way are, however, low because of the low oil-solubility of the active ingredients.

Also known are a number of processes in which initially a fine-particle dispersion of the poorly soluble substances in an aqueous medium is produced. This dispersion is then converted by removal of the medium into a fine-particle dry powder of the substances, see WO 94/01090, WO 93/10768, EP 239949, EP 425892, DE 37 42 473, etc. Accordingly, EP 0 065 193 A2 also discloses a process for producing carotenoid and retinoid products in powder form, in which the poorly soluble compound is rapidly dissolved in a volatile, water-miscible, organic solvent at elevated temperature, the poorly soluble compound is immediately precipitated in colloidal form from the resulting molecular solution by rapid mixing with an aqueous solution of a swellable colloid, and the resulting dispersion is freed of the solvent and the dispersing medium.

DE 37 02 030 A1 discloses a process for producing water-dispersible carotenoid preparations which are in powder form and in which the carotenoid is dissolved in an edible oil and the oily solution is present in the form of small droplets. In this case, the carotenoid is rapidly dissolved in a volatile, water-miscible, organic solvent at elevated temperature together with 1.5 to 20 times the amount by weight, based on the carotenoid, of an edible oil, and with an emulsifier, and then a two-phase mixture in which the oil is present as microdisperse phase with carotenoid dissolved therein is formed from the resulting molecular solution by immediate mixing with an aqueous solution of a protective colloid. The carotenoid preparation which is in powder form and which is obtained after removal of solvent and water contains the carotenoid dissolved in the edible oil, and the oily solution is dispersed in the form of small droplets in the protective colloid matrix in powder form.

Further processes such as the processes disclosed in EP 0 065 193 A2 and DE 37 02 030 A1 lead to redispersible dry powders, but also have some disadvantages. The colloidal solutions formed are very dilute, i.e. typical solids concentrations in these colloidal solutions are in the range from 0.5 to a maximum of 3% by weight. This means that to produce the powder required for a medicament or another product, e.g. a food coloring agent, it is necessary to remove a considerable quantity of solvent, in particular essentially water. The drying process most suitable for producing such powders is spray drying, which can be carried out well on the laboratory scale. However, no production which even approaches being economic is possible on the manufacturing scale. To produce only 100 kg of spray-dried powder it is necessary in the case of a colloidal solution having a total solids content of 3% by weight to spray dry more than 3000 l of colloidal solution.

A further disadvantage is that the particles present in the colloidal solutions tend to agglomerate during storage of the solutions, resulting in particles of increasing size, which eventually sediment. This means that the colloidal solutions must be dried rapidly, without intermediate storage. An on-line variant in which the colloidal solutions with a solids content of from 1 to 3% by weight are immediately dried directly after their production however requires in the preferred process of spray drying a very large and thus uneconomic spraying capacity.

An additional disadvantage is that the protective colloids typically used are natural substances or natural substance derivatives, such as, for example, casein or gelatin, whose aqueous solutions are subject to rapid microbial attack. For this reason too it is not possible to store the colloidal solutions of the poorly soluble compounds over a prolonged period, except where appropriate in the case of elaborate microbe-free working and/or on addition of preservatives to reduce microbes.

It has emerged that conventional processes for increasing the solids content of a dispersion have disadvantages. The disadvantage of the centrifugation process is, for example, that the low particle concentration and the small particle size in the present invention require long processing times and high centrifugal forces.

Conventional (dia)filtration cannot be used because, in the case of the present invention in which the poorly soluble compounds are present in colloidal dispersion, the filter layer becomes covered progressively over the processing time with the colloidal material which has been filtered off, resulting in slow blockage of the filters. In addition, very high particle concentrations result in the colloid layer deposited on the filter surface, which considerably favor unwanted and irreversible particle agglomeration.

The removal of liquid medium by distillation to increase the solids content has also proved disadvantageous, on the one hand because it represents an energy-expending process which must take place at elevated temperature and/or with reduced pressure, and on the other hand because the dispersed poorly soluble compound may be harmed by the thermal stress. A crucial disadvantage of all processes based on evaporation of liquid is moreover that in this case only the liquid itself, but not the substances dissolved therein, are removed. Slight, unavoidable impurities may therefore be highly enriched in the final product. With a solids concentration of 1% in the dispersion there is enrichment of the impurities by a factor of 100 in a spray-dried final product. If the dispersion contains different dispersants or solvents, distillation at different speeds may occur on evaporation, resulting in changes in the dispersant/solvent composition in the meantime, which may be disadvantageous for the stability of the colloidal dispersion of the poorly soluble compound.

WO 96/35414 describes in the examples a process for producing nanoparticles of a poorly soluble active ingredient using a cross-flow filtration. This filtration is, however, used not for concentration but for purification of the dispersion, with a considerable increase in volume.

The problems which have been mentioned make it clear that, despite the advantages of the formulations described with the previously disclosed processes, economic production of water-dispersible dry powders of poorly soluble compounds is not possible on the manufacturing scale.

It is an object of the present invention to provide a process for producing water-dispersible dry powders of poorly soluble compounds which avoids the disadvantages of the prior art.

It should in particular be possible to manage the process in such a way that prolonged storage times of unstable or readily spoiled solutions or dispersions of the poorly soluble compounds are avoided.

The process should additionally permit economic production of the water-dispersible dry powders.

We have now found, surprisingly, that the process of tangential filtration or “cross-flow filtration” is particularly suitable for concentrating the dispersions which contain the poorly soluble compounds in colloidal form within the necessary constraints of economics, short processing times, avoidance of microbial attack and avoidance of agglomeration. All the disadvantages of distillation processes and substantially also the disadvantages of diafiltration processes are avoided on use of this process.

The invention therefore relates to a process for producing water-dispersible dry powders of poorly water-soluble compounds, which comprises the following steps:
a) production of a dispersion which comprises the poorly soluble compound in microdisperse form in a dispersant
b) concentration of the dispersion of the poorly soluble compound by tangential filtration and
c) removal of the remaining dispersant.

The present invention also relates to a preparation based on a water-dispersible dry powder of poorly water-soluble compounds, where the water-dispersible dry powder is obtainable by the process of the invention.

The concentration of the dispersion before removal of the dispersant results in a reduction in the amount of dispersant which must be removed with expenditure of time and energy. This shortens the time expended on the removal of dispersants such that it is possible for produced dispersions to be immediately dried, without intermediate storage and without the need for drying apparatuses of uneconomically large dimensions.

Step a) of the process of the invention, the production of a dispersion comprising the poorly soluble compound in microdisperse form in a dispersant, can in principle be carried out in any way. Numerous processes for producing such a dispersion are described, see the prior art cited at the outset. However, it is preferred to produce the dispersion by the process called mixing chamber micronization as described, for example, in EP 0 065 193 A2 or in DE 37 02 030 A1. The disclosure content of these applications, in particular in relation to process management, in relation to the solvents or dispersants used, the protective colloids and other additions, and in relation to the concentrations and ratios of the compounds used to one another, is hereby incorporated in the present invention by reference. Accordingly, the dispersion comprising the poorly soluble compound in microdisperse form is preferably produced according to the invention by dissolving the poorly soluble compound in a volatile, water-miscible, organic solvent at temperatures between 50 and 200° C., where appropriate under elevated pressure, within a time of less than 10 s, and immediately precipitating the poorly soluble compound in colloidal form from the resulting molecular solution by rapid mixing with an aqueous solution of a swellable colloid at temperatures between 0° C. and 50° C. Thus, in this case, the poorly soluble compound is present in the form of microdisperse particles in a dispersant which consists of the volatile, water-miscible, organic solvent and of water.

Alternatively, the disperson comprising the poorly soluble compound in microdisperse form is preferably produced by rapidly dissolving the poorly soluble compound in a volatile, water-miscible, organic solvent at temperatures between 50 and 240° C., together with 1.5 to 20 times the amount by weight, based on the poorly soluble compound, of an edible oil, and with an emulsifier, where appropriate under elevated pressure, and transferring the hydrophilic solvent component from the resulting molecular solution into the aqueous phase by immediate mixing with an aqueous solution of a protective colloid at temperatures between 0° C. and 50° C., where the hydrophobic oil phase containing the dissolved poorly soluble compound results as microdisperse phase. Thus, in this case, the dispersion is a two-phase mixture with oil particles as microdisperse particles. The poorly soluble compound is present in solution in the oil particles. The dispersant consists of the volatile, water-miscible, organic solvent and water.

Preferred water-miscible volatile solvents are alcohols, ketones, esters, acetals and ethers, especially acetone, 1,2-butanediol 1-methyl ether, 1,2-propanediol 1-n-propyl ether, ethanol, n-propanol, isopropanol and mixtures thereof.

Suitable protective colloids are any protective colloids approved for the purpose of use, for example gelatin, starch, dextran, pectin, gum arabic, casein, caseinate, whole milk, skimmed milk, milk powder or mixtures thereof. Polyvinyl alcohol, polyvinylpyrrolidone, methylcellulose, carboxymethyl-cellulose, hydroxypropylcellulose and alginates are also preferred colloids.

It is possible in addition to add plasticizers, for example sugars or sugar alcohols, to the colloid to increase the mechanical stability of the final product. It is moreover possible to add preservatives and/or oxidation stabilizers as required. Suitable compounds are in each case mentioned in the abovementioned patent applications. Suitable edible oils are in particular oils which are liquid at 20 to 40° C. Examples are vegetable oils such as corn oil, coconut oil, sesame oil, arachis oil, soybean oil or cottonseed oil. Other suitable oils or fats are lard, beef tallow and butter fat. The edible oils are generally used in 1.5 to 20 times, preferably 3 to 8 times, the amount by weight based on the poorly soluble compound, and the total oil content of the preparation of the poorly soluble compound should not exceed 60% by weight if a dry powder is to be produced.

A suitable apparatus for producing the dispersions is likewise described in EP 065 193 A2 and DE 37 02 030 A1.

The particles of the dispersion in stage a) generally have a size in the range from 0.01 to 100 μm, in particular 0.02 to 10 μm. Particularly preferred dispersions are those in which the dispersed particles have average particle sizes of from 0.01 to 5 μm, preferably 0.05 to 0.8 μm. These can be obtained for example as described in EP 065 193, EP 239 949, EP 425 892 or DE 37 02 030. If the poorly soluble compound as such is in the form of a colloidal dispersion, the dispersed particles are ordinarily smaller than when the poorly soluble compound is dissolved in dispersed oil droplets. However, the process of the invention is not confined to compounds having these particle sizes.

Dispersions comprising a poorly soluble compound in colloidal form can be produced only with low solids contents. If no concentration of the dispersion is carried out, the content of poorly soluble compound is typically 1 to 3% by weight. However, the present invention is not confined to dispersions having these solids contents but has advantages also where the solids contents are higher, above all, of course, at solids contents below 1% by weight.

The poorly water-soluble compounds are preferably those having a solubility of ≦10 g/l, in particular ≦5 g/l and particularly preferably ≦1 g/l of water (at 25° C.).

Poorly water-soluble compounds may be organic or inorganic compounds. Preference is given to pharmaceutical, dietary, cosmetic and pesticidal active ingredients, there being no restriction whatsoever in relation to the chemical type. Active pharmaceutical ingredients include hormones, vitamins, provitamins, enzymes, phytopharmaceuticals and plant extracts. Examples of preferred active ingredient groups and active ingredients are:
analgesics/antirheumatics such as codeine, diclofenac, fentanyl, hydromorphone, ibuprofen, indomethacin, levomethadone, morphine, naproxen, piritramide, piroxicam, tramadol
antiallergics such as astemizole, dimetindene, doxylamine, loratadine, meclozine, pheniramine, terfenadine
antibiotics/chemotherapeutics such as erythromycin, framycetin, fusidic acid, rifampicin, tetracycline, thiacetazone, tyrothricin
antiepileptics such as carbamazepine, clonazepam, mesuximide, phenytoin, valproic acid
antimycotics such as clotrimazole, fluconazole, itraconazole
calcium channel blockers such as darodipine, isradipine
corticoids such as aldosterone, betametasone, budesonide, dexamethasone, fluocortolone, fludrocortisone, hydroxycortisone, methylprednisolone, prednisolone
hypnotics/sedatives benzodiazepines, cyclobarbital, methaqualone, phenobarbital
immunosuppressants azathioprine, cyclosporin
local anesthetics benzocaine, butanilacaine, etidocaine, lidocaine, oxybuprocaine, tetracaine
migrane remedies dihydroergotamine, ergotamine, lisuride, methysergide
anesthetics droperidol, etomidate, fentanyl, ketamine, methohexital, propofol, thiopental
opthalmologicals acetazolamide, betaxolol, bupranolol, carbachol, carteolol, cyclodrine, cyclopentolate, diclofenamide, edoxudine, homatropine, levobunolol, pholedrine, pindolol, timolol, tropicamide
phytopharmaceuticals hypericum, urtica folia, artichoke, agnus castus, cimicifuga, devil's claw, broom, peppermint oil, eucalyptus, celandine, ivy, kava-kava, echinacea, valerian, palmetto, milk thistle, Ginkgo biloba, Aloe barbadensis, Allium sativum, Panax ginseng, Serenoa repens, Hydrastis canadensis, Vaccinium macrocarpon or mixtures thereof
protease inhibitors e.g. saquinavir, indinavir, ritonavir, nelfinavir, palinavir, tipranavir or combinations of these protease inhibitors
sex hormones and their antagonists anabolics, androgens, antiandrogens, estradiols, progestins, progesterone, estrogens, antiestrogens such as tamoxifen
vitamins, provitamins, antioxidants such as carotenoid or carotenoid analogs, e.g. β-carotene, canthaxanthin, astaxanthin, lycopene or lipoic acid, vitamin A, vitamin Q
cytostatics/antimetastatics busulfan, carmustin, chlorambucil, cyclophosphamide, dacarbazine, dactinomycin, estramustine, etoposide, flurouracil, ifosfamide, methotrexate, paclitaxel, vinblastine, vincristine, vindesine.

The dispersion obtained in step a) is concentrated according to the invention by tangential filtration (step b)), with the solids content after the concentration preferably being 1 to 20% by weight. Tangential filtration is a screen filtration process which is known per se and in which, in contrast to diafiltration, the medium to be filtered is not forced directly onto the filter layer in order to form a filter cake there, but is kept in continuous motion. The term dynamic filtration is also used because of the continuous motion of the medium to be filtered. Formation of a filter cake is prevented or at least greatly delayed because the filter medium, i.e. the filtration surface, is continuously washed clean. The motion of the medium to be filtered can be achieved by continuous circulation of this medium using a pump, or it is possible to use a filter designed so that the medium to be filtered can continuously flow through it and is completely or sufficiently freed of liquid medium on its way through the filter.

The filtration process takes place on membranes whose pore sizes are to be selected in accordance with the particle sizes of the particles to be removed. When the particles to be removed have a particle size of about 0.01 μm to about 0.1 μm, the term used is ultrafiltration, and when the particles to be removed have a particle size of about 0.1 μm to about 10 μm it is microfiltration. The process is therefore very suitable for retaining colloidal particles, i.e. for concentrating colloidal dispersions.

The membranes for microfiltration and ultrafiltration are generally, for mechanical reasons, applied to a monolayer or multilayer substructure as support made of the same or different material as the membrane. The separation layers may consist of organic polymers, ceramic, metal or carbon. The membranes are in practice incorporated into so-called membrane modules. Module geometries suitable in this connection are those which are mechanically stable under the temperature and pressure conditions of the filtration. Suitable examples are flat, tubular, multichannel element, capillary or coiled geometry.

To increase the filtration efficiency, the tangential filtration is normally operated as pressure filtration, with the pressure typically being in the range from 0.2 to 1 MPa. The flow rates are typically about 2 to 4 m/s, and the permeate rates may be, depending on the pore size and filtration pressure, up to 3000 l per m² of filter membrane and hour.

The concentrating in step b) represents a step in an overall process and it is therefore desirable for the process times necessary therefor to be reproducible and reliably predictable. Conventional filtration processes are associated with imponderables since the filtration rate decreases to a greater or lesser extent through the formation of a filter cake and the blockage of the filter pores. In tangential filtration by contrast the amount of liquid separated through the membrane remains substantially constant over the process time, and blocking of the filter pores is likewise counteracted. Further advantages are that the process can be carried out under very mild conditions, thus counteracting possible particle growth. It is possible in addition to operate in closed systems, and even microbe-free if necessary, which may be desirable in respect of protective colloids which are susceptible to microbial attack.

It has emerged that membranes particularly suitable in the present invention for concentrating the colloid dispersions are made of polyethersulfone or regenerated cellulose, as are available for example from Millipore under the name BIOMAX (polyethersulfone) and ULTRACEL. However, it is equally possible to use membranes from other manufacturers and membranes produced from other materials, e.g. those typically employed for ultrafiltration. The filter membranes are available in various filter pore sizes. Filter membranes suitable for the concentrating in the process of the invention are therefore in particular those having a molecular weight exclusion limit above about MW 100,000, i.e. particles above this molecular weight are held back by the membrane and remain in the concentrated colloid dispersion, i.e. in the retentate. Membranes with MW exclusion limits of from 500 000 to 1 000 000 are preferred.

The tangential filtration can be adjusted very specifically to the colloidal solution to be concentrated in each case, because a large number of different filter membranes are available on the market, so that virtually any desired filter pore size and any desired filter material are available. The filter membranes are standardized and obtainable in constant quality. The membranes are commercially available as ready-to-use filtration unit, i.e. the filter membrane is incorporated into a metal or plastic housing which has both connections for the colloidal solution to be concentrated and an outlet for the filtered liquid (filtrate). Corresponding complete apparatuses are commercially available from the laboratory scale to the manufacturing scale, appropriate for the respective tasks.

A particular embodiment of the present invention is the combination of concentrating the colloidal dispersions by tangential filtration with procedures for reversible enlargement of the colloidal particles. A greater difference in molecular weight between constituents to be removed and particles to be retained means that they can be separated from one another with fewer problems. It is therefore advantageous for the poorly soluble compounds which are present in colloidal form to be reversibly associated to give larger aggregates before the tangential filtration. It is then possible to choose filter membranes with larger pore diameters, thus considerably increasing the filtration rate.

Various processes are possible for reversible agglomeration of the colloidal particles, e.g. through addition of inorganic and/or organic salts (“salting out”), by increasing or reducing the temperature, or by changing the pH of the colloidal dispersion. Combinations of these processes are also possible.

It is possible in this way to form by the reversible agglomeration from the original colloidal particles, which are preferably in the size range of about 50 to 800 nm, aggregates in the size range from micrometers to millimeters. Very coarse-pore membranes displaying a high filtration rate are then sufficient for the concentration.

The agglomeration must be reversible, i.e. the original particle size distribution of the poorly soluble compounds in the colloidal dispersion before the agglomeration must be restorable. It is possible in the individual cases to establish by routine experiments which of the abovementioned processes is suitable. On use of ionic protective colloids such as, for example, casein, it is appropriate to change the pH. This anionic protective colloid is soluble or colloidally soluble only at neutral and weakly basic pH values. In an acidic pH environment there is protonation of the carboxyl function of the casein, resulting in precipitation/flocculation. This process can be reversed by increasing the pH. Preparations of poorly soluble compounds produced using casein as protective colloid can therefore easily be precipitated by reducing the pH and can in this state be concentrated very efficiently, i.e. rapidly. After removal of the desired amount of solvent it is then possible to increase the pH again, thus obtaining the original colloidal dispersion again.

In the case of nonionic protective colloids, other processes are preferred for reversible agglomeration, e.g. the addition of concentrated salt solutions or the addition of a water-soluble salt itself.

Processes for the agglomeration of colloidal dispersions are known in the art and need to be checked for their reversibility only in the individual case. The dispersion can be dried after the redispersion of the agglomerated particles.

It is possible in the process of the invention to avoid the colloidal dispersions standing for prolonged times before drying by adapting the throughput of the tangential filtration unit to the amount of colloidal dispersion prepared per unit time. It is possible to concentrate the prepared amount of colloidal dispersion directly without intermediate storage and pass it on for drying without further intermediate storage. This is especially advantageous when the dispersion has insufficient storage stability after the concentration or even before that.

The process of the invention can be carried out batchwise, semicontinuously or continuously. A possible process is therefore one in which one batch of an initial dispersion is produced, this batch is concentrated directly after production, and the concentrated batch is freed of dispersant immediately after the desired concentration is reached, i.e. the individual steps of the process of the invention can be carried out batchwise. It is possible alternatively for the individual steps themselves to be carried out continuously, i.e. for example the initial dispersion can be produced continuously or batchwise and passed on continuously to a tangential filtration unit and, after the desired concentration, to a drying apparatus. A tangential filtration unit which is preferred for this purpose is designed so that the necessary concentration is achieved on flowing through the filtration unit once.

A dry powder can be prepared from the concentrated dispersion in a conventional way, e.g. as disclosed in DE-OS 25 34 091, by spray drying, removal of the particles or drying in a fluidized bed. The preferred drying process is spray drying. The concentrated dispersion can be spray dried without further pretreatment such as, for example, stripping off solvent by distillation, i.e. all the dispersant still present is stripped off in the spray tower. The water-dispersible dry powder ordinarily results in dry and free-flowing form at the base of the spray tower. It may be expedient where appropriate for a powder which has been only partially dried by spray drying to be completely dried in a fluidized bed.

The present invention is described in more detail below by examples which are to be regarded as explanatory and not restrictive.

### EXAMPLE 1

A water-dispersible dry powder containing 35.7% by weight of coenzyme Q10 and 64.3% by weight of casein was produced.

Firstly, an aqueous colloidal solution of the stated ingredients was produced by mixing chamber micronization as described in EP-0 065 193 A2. The colloidal solution had (before the concentration) a coenzyme Q10 active ingredient content of 0.6% by mass and a particle size distribution with a center of gravity at about 200 nm, all the particles being smaller than 1 μm. This distribution was also present unchanged after storage of the solution for 24 hours, i.e. the solution was relatively storage-stable.

This colloidal solution was concentrated by tangential filtration, the conditions being as follows:

| Initial conditions: | | |
|---|---|---|
| Initial volume: | about 2.5 | l |

| | |
|---|---|
| Temperature: | room temperature |
| Process conditions: | |

| | | | |
|---|---|---|---|
| Membrane: | Ultracel ® | 0.1 | m² |
| | (Millipore GmbH, Eschborn) | | |
| | 100 kD V screen, area: | | |

| Feed pressure: | 0.6 | bar |
|---|---|---|
| Retentate pressure: | 0.2 | bar |
| Trans-channel pressure drop (dP) | | |
| (function of the cross flow) : | 0.4 | bar |
| Trans-membrane pressure (TMP) : | 0.4 | bar |
| Cross flow: | 14 | l/min/m² |
| Initial flow rate at t₀: | 34 | l/h/m² |
| Final flow rate at t_{final}: | 8 | l/h/m² |
| Average overall flow rate: | 16 | l/h/m² |
| Total concentration time (t₀ → t_{final}): | 125 | mm |
| TMP and dP were kept constant | | |
| throughout | | |
| the concentration process. | | |
| Temperature: | room | |
| | temperature | |
| Active ingredient concentration | <0.07% | (m/m) |
| in the eluate: | | |
| Properties of the concentrate: | | |
| Final volume: | about 0.25 | l |
| Temperature: | room temperature | |
| Active ingredient concentration: | 7.1% | (m/m) |
| Concentration factor: | about 12 | |

This means that only very little active ingredient was removed from the colloidal solution through the membrane.

The membrane which was used is easy to clean. Rinsing with 0.1 N NaOH (about 10 min) at room temperature led to virtually complete restoration of the initial state (92.7% of the original NWP=normalized water permeability). This means that little or no product penetrates into the membrane and only relatively little of it is able to adsorb on the membrane.

Formulation A can thus be concentrated by a factor of 12 under mild conditions in a relatively short process time without the need to accept significant losses of product during this.

### EXAMPLE 2

A water-dispersible dry powder of the following composition was produced:

| Ingredient | Mass [% (w/w)] |
|---|---|
| β-Carotene | 11.0 |
| Ascorbyl palmitate | 1.0 |
| α-Tocopherol | 2.0 |
| Gelatin B100 | 5.0 |
| GelitaSol P (gelatin hydrolysate) | 25.0 |
| Lactose | 52.0 |
| Water (residual moisture) | 4.0 |

An aqueous colloidal dispersion containing the above ingredients was produced in analogy to example 1. The β-carotene active ingredient content (before concentration) was 1.1% by mass. The particle size distribution was bimodal. Some of the particles had a diameter below 1 μm, and the center of gravity of the distribution in this case was at about 200 nm. The other center of gravity of the particle size distribution was at about 16 μm, with the particle diameter being less than 20 μm. This distribution was still present unchanged after the solution had been stored for 24 hours, i.e. the solution was relatively stable on storage.

Conditions for the concentration by tangential flow filtration:

| Initial conditions: | | | |
|---|---|---|---|
| Initial volume: | | about 5.0 | l |
| Temperature: | | room temperature | |
| Process conditions: | | | |
| Membrane: | Ultracel ® | 0.1 | m² |
| | (Millipore GmbH, | | |
| | Eschborn) | | |
| | 100 kD V screen, area: | | |

| Feed pressure: | 0.6 | bar |
|---|---|---|
| Retentate pressure: | 0.1 | bar |
| Trans-channel pressure drop (dP) | | |
| (function of the cross flow) : | 0.5 | bar |
| Trans-membrane pressure (TMP) : | 0.35 | bar |
| Cross flow: | 16 | l/min/m² |
| | 97 | 1/h/m² |
| | 30 | l/h/m² |
| | 77 | l/h/m² |
| Total concentration time (t₀ →t_{final}): | 34 | mm |
| TMP and dP were kept constant throughout | | |
| the concentration process. | | |
| Temperature: | room temperature | |
| Active ingredient concentration | <0.001% | (m/m) |
| in the eluate: | | |
| Properties of the concentrate: | | |
| Final volume: | about 0.25 | l |
| Temperature: | room temperature | |
| Active ingredient concentration: | 21.3% | (m/m) |
| Concentration factor: | about 20 | |

This means that only very little active ingredient was removed from the colloidal solution through the membrane.

Particle Size Distribution:

No changes in the particle size distribution were detectable after the concentration compared with the state before the concentration process.

The dispersion can thus be concentrated by a factor of 20 under mild conditions in a very short process time without suffering harm and without the need to accept significant losses of product. It is possible to produce 10 l of concentrate from 200 l of initial solution within 3 hours. A membrane area of 1 m² is required for this. The membrane which was used is easy to clean: simply rinsing with water (for about 10 min) at room temperature leads to a virtually complete restoration of the initial state (93.7% of the original NWP=normalized water permeability). This means that little or no product penetrates into the membrane and only little of it is able to adsorb onto the membrane.

### EXAMPLE 4

A yellowish colloidal active ingredient-containing solution having a total solids content of 0.5% by weight was produced in analogy to example 1 with the protective colloid casein (65% by weight) and the active ingredient coenzyme Q10 (35% by weight). This solution was then acidified to pH=1 by stepwise addition of aqueous hydrochloric acid (2 mol/l), causing complete flocculation of the solids in the colloidal solution. It was possible to filter this precipitate off by vacuum filtration (paper filter membrane or glass frit); the filtrate was colorless. This removed precipitate was then dispersed again with stirring at room temperature in dilute sodium hydroxide solution (0.1 mol/l) in a concentration of 0.5% total solids content, resulting in a yellowish colloidal solution again. It was then possible to adjust this alkaline colloidal solution to a pH of 7 using small amounts of hydrochloric acid without flocculation.

The particle size distribitions of the resulting colloidal solutions were measured using a Malvern Mastersizer particle size measuring instrument. The initial solution before the acidification with HCl showed an average particle size of 0.2 μm (90% below 0.4 μm); no particles above 1 μm were detectable. The average of the particle size distribution of the precipitate formed with HCl and redispersed again in dilute NaOH was 0.3 μm (90% below 0.5 μm), and only about 1.5% of the particles were above 1 micrometer.

We claim:

1. A process for producing water-dispersible dry powders of poorly water-soluble compounds, which comprises the following steps:
a) production of a dispersion which comprises 0.5 to 3% by weight of the poorly soluble compound in microdisperse form as well as a protective colloid in a dispersant,
b) concentration of the dispersion of the poorly soluble compound to the 10- to 40-fold solid content by tangential filtration, thereby obtaining a concentrated dispersion and
c) removal of the remaining dispersant from the concentrated dispersion.

2. A process as claimed in claim 1, in which a dispersant consisting of water and a volatile, water-miscible, organic solvent is used.

3. A process as claimed in any of the preceding claims, in which a dispersion is produced, in which the dispersed particles have particle sizes of from 0.01 to 5 μm, preferably 0.05 to 0.8 μm.

4. A process as claimed in any of the preceding claims, in which a filter membrane made of polyethersulfone or regenerated cellulose is used in the tangential filtration.

5. A process as claimed in any of the preceding claims, in which a filter membrane with a molecular weight exclusion limit above 100 000, preferably from 500 000 to 1 000 000, is used in the tangential filtration.

6. A process as claimed in any of the preceding claims, in which the removal of the dispersant takes place by spray drying.

7. A process as claimed in any of the preceding claims, in which the production of the dispersion, the concentration of the dispersion and the removal of the dispersant take place continuously.

8. A process as claimed in any of the preceding claims, in which the microdispersed particles present in the dispersion are reversibly agglomerated before the concentration and microdispersed after the concentration.

9. A process as claimed in claim 9, in which the dispersed particles are agglomerated by
addition of inorganic and/or organic salts, and/or
changing the temperature of the dispersion, and/or
changing the pH of the dispersion.
